(19) United States
(12) Patent Application Publication
(10) Pub. No.: US 2022/0016354 A1
(43) Pub. Date: Jan. 20, 2022

(54) DEVICE AND METHOD FOR IDENTIFYING INJECTOR ACTIVITIES

(71) Applicant: InsuLog Ltd., Ramat-Gan (IL)

(72) Inventor: Menash MICHAEL, Hofit (IL)

(73) Assignee: InsuLog Ltd., Ramat-Gan (IL)

(21) Appl. No.: 17/298,579

(22) PCT Filed: Nov. 28, 2019
(86) PCT No.: PCT/IL2019/051309

§ 371 Date: May 30, 2021

Related U.S. Application Data
(60) Provisional application No. 62/772,651, filed on Nov. 29, 2018.

(51) Int. Cl. A61M 5/315

(52) U.S. Cl. CPC A61M 5/31568; A61M 2205/3327; A61M 2205/3584; A61M 5/3155

(57) ABSTRACT

A method and system for detecting rotational movements due to dosage changing activity in a pen-type injector having a body and a dosage adjustment mechanism. The method includes measuring, using a sensor, rotation about one or more axes of the injector to provide rotational data, identifying in the rotational data, one or more features and/or patterns corresponding to a change in the angular velocity of the sensor due to mechanical coupling between the body and the dosage adjustment mechanism, and detecting the rotational movements based on the identifying.

### RELATED APPLICATION

The present application claims the benefit of priority and under 119(e) of U.S. Provisional Patent Application No. 62/772,651 filed on 29 Nov. 2018, the disclosure of which is incorporated herein by reference.

### FIELD AND BACKGROUND

The present invention, in some embodiments thereof, relates to identifying activities of injectors, and more particularly, but not exclusively, to identifying activities of injectors in accordance to movements of the injector.

U.S. Patent Application Publication No. 2017/0182258 discloses “an adjunct device for tracks time and/or dosage of a medicine. The device may include a connector for mounting the device to a deposable pen injector. The device may be configured to allow use of the native controls and injectors of the injector. For example, the device may include a view port for viewing a dose indicator of the injector. The device may include one or more vibration sensors. A processor may be configured to differentiate increasing a dose, decreasing a dose and/or discharging the medicine based on the output of the sensors. Optionally a display of the device may be positioned for simultaneous viewing with the dosage indicator of the injector. For example, a user may verify the accuracy of the adjunct device before performing a discharge”.

Additional background art includes “U.S. Provisional Patent Application No. 62/272,275” and “EP Patent Application Publication No. 3188061”.

### SUMMARY

According to an aspect of some embodiments of the present invention there is provided a method for detecting rotational movements due to dosage changing activity in a pen-type injector having a body and a dosage adjustment mechanism.

According to some embodiments of the invention, the method comprises measuring, using a sensor, rotation about one or more axes of the injector to provide rotational data, identifying in the rotational data, one or more features and/or patterns corresponding to a change in the angular velocity of the sensor due to mechanical coupling between the body and the dosage adjustment mechanism, and detecting the rotational movements based on the identifying. In some embodiments of the invention, the change in the angular velocity is a sudden change, having a sudden angular acceleration.

According to some embodiments of the invention, the one or more features comprise rotational data about a first axis having an angular velocity profile that is distinct from angular velocity profile about the other axes.

According to some embodiments of the invention, the method comprises classifying the one or more features as dosage change when having an angular velocity higher than a threshold during an increase of the velocity or at the peak velocity. In some embodiments, classifying the one or more features as dosage change is by having a positive angular velocity duration lower than a threshold. In some embodiments, classifying the one or more patterns as dosage increase is by having a positive peak angular velocity higher than a threshold followed by a decreasing of the angular velocity to a negative rate. In some embodiments, classifying the one or more patterns as dosage decrease is by having a peak negative angular velocity lower than a threshold followed by an increase in the angular velocity to a positive rate. In some embodiments, classifying the one or more features as an injection operation is by having a positive angular velocity duration lower than a threshold. In some embodiments, differentiating between patterns of rotational movements and patterns of linear movements is by classifying a pattern of linear movements as absent of distinct rotational movements about one axis. In some embodiments, classifying the one or more patterns as a dosage adjustment is by a ratio between a peak positive angular velocity to a peak negative angular velocity.

According to some embodiments of the invention, the measuring is about one axis. In some embodiments, the measuring is by one or more sensors disposed at an add-on sensing device connected to the injector. In some embodiments, the one or more sensors comprise a gyro sensor. In some embodiments, the measuring is by one sensor.

According to an aspect of some embodiments of the present invention there is provided a dosage change indication system for an adjustable pen-type injector having a mechanical coupling between a dosage adjustment mechanism and the injector.

According to some embodiments of the invention, the system comprises one or more sensors orientated to measure rotations about one or more axes of the injector, and to provide a rotational data. In some embodiments, the system comprises one or more processors having a rotational data receiving functionality. In some embodiments, the one or more processors have a dosage adjustment identification functionality operatively connected to the rotational data receiving functionality to identify in the rotational data, one or more features and/or patterns corresponding to a change in the angular velocity of the sensor due to the mechanical coupling. In some embodiments, the one or more sensors are configured to measure rotational movements about an axis, which is the axis of the rotational movements produced by the mechanical coupling.

According to some embodiments of the invention, the system comprises an add-on sensing device having one or more connection surfaces, and the one or more sensors are disposed within the sensing device. In some embodiments, the one or more connection surfaces are urged against the injector, when the add-on sensing device is connected to the injector. In some embodiments, the injector comprises a body and a dosage dial, and the add-on sensing device comprises a housing connected to the body, when the sensing device is connected to the injector. In some embodiments, the add-on sensing device comprises a housing connected to the dosage dial, when the sensing device is connected to the injector. In some embodiments, the add-on sensing device comprises the processor.

According to some embodiments of the invention, the system comprises a wireless communication circuit and the processor receives sensor measurements through wireless communication.

According to some embodiments of the invention, the one or more sensors comprise a gyro sensor. In some embodiments, the system comprises a single sensor to measure the rotational movements.

According to some embodiments of the invention, the system comprises one or more body holders having a holding surface and one or more cushion layers disposed between the holding surface and the injector, when the body holder is connected to the injector.

According to some embodiments of the invention, the system comprises a computing device other than the add-on sensing device or the injector, and the processor is disposed in the computing device.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out activities for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert is not capable of processing rotational data signals in the ways a computer can/does, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1a and 1b are simplified illustrations of example dosage changing activities performed by injectors.

FIG. 2 is a is a simplified illustration of a side view and a top view of an injector detection system, according to some embodiments of the invention;

FIG. 2a is a is a simplified side view illustration of an injector detection system, according to some embodiments of the invention;

FIG. 2b is a is a simplified side view illustration of an injector detection system, according to some embodiments of the invention;

FIG. 3 is a is a simplified side view illustration of an injector detection system, according to some embodiments of the invention;

FIG. 4 is a simplified illustration of a diagram of a detection process, according to some embodiments of the invention.

FIGS. 5 to 10 are 2D graphs that illustrate example measurements of rotational motions about axes X, Y, and Z, according to some embodiments of the invention;

FIG. 11 is a simplified side view illustration of an injector detection system, according to an embodiment of the invention;

FIG. 12 is simplified side view illustration of an injector detection system, according to an embodiment of the invention.

FIG. 13 is a simplified side view illustration of an injector detection system, according to an embodiment of the invention.

FIGS. 14a and 14c are simplified perspective view illustrations of an add-on sensing device, according to some embodiments of the invention.

FIG. 14b is a simplified side view illustration of an add-on sensing device, according to some embodiments of the invention; and

FIG. 15 is a simplified illustration of a diagram of a calibration process, according to some embodiments of the invention.

### DESCRIPTION OF SPECIFIC EMBODIMENTS OF THE INVENTION

The present invention, in some embodiments thereof, relates to identifying activities of injectors, and more particularly, but not exclusively, to identifying activities of injectors in accordance to movements of the injector.

### OVERVIEW

Some pen-type injectors use a mechanical interface, having a mechanical coupling between the body of the injector and the dosage adjustment mechanism, and produces interruptions in the movements of the dosage adjustment mechanism. The interruptions during a dosage adjustment may result in “clicking” sounds, by which the operator receives a feedback about the increasing or the decreasing a medicine dosage, and/or dosage injection. In some cases, a “click”, would indicate that a unit of dosage has been added or reduced. A “click” can be an indication of a natural stopping place for the adjustment.

According to some embodiments, the interruptions in the movement result in sudden rotational motions of the mechanical interface. In some embodiments, a sudden motion is a change in angular velocity. In some embodiments, a sudden motion is a change in the angular acceleration. In some embodiments, a sudden motion is a change in the angular acceleration rate.

An aspect of some embodiments of the invention relates to detecting rotational movements due to dosage changing activity in an injector by measuring, using a sensor, rotation of the injector to provide rotational data. In some embodiments of the invention, the sensing is used to detect rotation about one or more axes of the injector. In some embodiments of the invention, a different axis, not aligned (e.g., not within +−20 degrees or 10 degrees of a device axis, such as the longitudinal axis) is used.

According to some embodiments, a mechanical coupling, between the body of the injector and the dosage adjustment mechanism the injector, produces sudden rotations. In some embodiments, detection is by identifying rotations data having a characteristic of a sudden rotational motion. In some embodiments, the identification is by one or more features and/or patterns of the rotational data.

In some embodiments, dosage changing activities are differentiated by classifying the rotational data as having features/patterns of one or more activities of the injector such as an increasing of dosage, a decreasing of dosage, and a dosage discharge (e.g. during injection).

According to some embodiments, features and/or patterns of rotations are distinct about a first axis, from the features and/or patterns of rotations about other axes. In some embodiments, the first axis is parallel to the axis of the rotation of the dosage dial. In some embodiments, measuring the rotation about the first axis is enough to detect rotational movements due to dosage changing activity.

According to some embodiments, the features and/or patterns are of the kinetics profiles of the rotations. For example the features and/or patterns may include: a peak angular velocity, a rate of reaching an angular velocity, a duration of angular velocity in a specified direction, a ratio between a peak angular velocity in one direction and a peak angular velocity in the opposite direction, a duration of the angular velocity in a positive and/or negative direction (positive is defined as in an increase of dosage and negative is defined as a decrease of dosage).

In some embodiments, the sensor is a gyro. In some embodiments, measuring the rotational motions is by using one sensor.

Some embodiments of the invention relate to a dosage change indication system having one or more sensors orientated to measure rotations about one or more axes of the injector, and one or more processors having a rotational data receiving functionality, and a dosage adjustment identification functionality operatively connected thereto. In some embodiments, the dosage adjustment identification functionality of the processors identifies, in the rotational data received from the sensors, features and/or patterns corresponding to rotational motions produced by the mechanical coupling. In some embodiments, the one or more of processors detect the dosage changing activities according to a classification of the motion features and/or patterns.

Some embodiments of the invention relate to a dosage change indication system having an add-on sensing device having one or more connection surfaces, configured to be attached to the injector to transmit rotational movements from the injector to the add-on sensing device. In some embodiments, the add-on sensing device has a housing with one or more of the connection surfaces. In some embodiments, the one or more sensors are disposed within the housing and orientated to measure rotational movements about one or more axes of the injector.

An aspect of some embodiments of the invention relates to preventing damping of the rotational data, used for detecting rotational movements. Some embodiments relate to having a body holder for preserving sensors signals measuring rotational motions of the sensors, when applying pressure on the body of the injector.

According to some embodiments, the dosage change indication system comprises one or more body holders having a holding surface and one or more cushion layers disposed between the holding surface and the injector, when the body holder is connected to the injector. In some embodiments, the body holder is an add-on. In some embodiments, the injector comprises the body holder. In some embodiments, the add-on sensing unit comprises the body holder.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Principles of Some Embodiments of the Invention

Patients using adjustable pen-type injectors need to inject a correct dosage and in the right schedule. The tracking of the injection time and the dosage requires manual steps, which may result in incorrect dosage setting and missing injection schedules.

One potential advantage of some embodiments of the invention is that the manual steps and confirmations are reduced by identifying dosage changing activities, for example by differentiating between rotational motions patterns related to dosage changing, and patterns unrelated to dosage changing (e.g. tapping the injector), and/or differentiating between rotational motions patterns relate to increasing dosage to patterns of decreasing of medical dosage.

In some adjustable pen-type injectors, such as insulin pens, a medicine dosage is adjustable by a rotating a mechanism (e.g. a dial), rotating in a first positive direction increases the dosage, and rotating in a second negative direction decreases it. Some injectors use a mechanical interface having a mechanical coupling between the body of the injector and the dosage adjustment mechanism, and produces interruptions in the movement of the dosage adjustment mechanism (e.g. set to correspond to a dosage unit). In some injectors, the mechanical interface applies to both rotational directions (positive and negative) of the dial.

According to some embodiment of the invention, a detection system is measuring and detecting the interruptions that result in sudden motion changes of the mechanical interface and/or changing the rotational velocity of the dosage dial. In some embodiment, the sudden motion is for a duration of 40 to 60 msec. In some embodiment, the sudden motion is for a duration of 20 to 50 msec. In some embodiment, the sudden motion is for a duration of 5 to 20 msec.

According to some embodiment of the invention, the sudden motion changes are rotational, having changes in the angular velocity (e.g., sudden acceleration) of the mechanism. In some embodiment, the sudden acceleration is for a period of less than 5 msec. In some embodiment, the sudden velocity change is 1 to 10 rad/sec in a duration of 1 to 5 msec. In some embodiment, the sudden velocity change is 2 to 5 rad/sec in a duration of 2 to 4 msec. In some embodiment, the sudden velocity change is 1 to 4 rad/sec in a duration of 1 to 4 msec. In some embodiment, the sudden motion comprises changes in the angular acceleration (jerk, optionally in the form of jerky motion) of the mechanism and/or the injector. Such jerk, can be on the order of, between 200 rad/sec\hat{ }2 and 10000 rad/sec\hat{ }2, for example, 200-1000, 1000-5000 and/or 5000-10000 rad/sec\hat{ }2 or smaller or intermediate or greater jerk values.

In some embodiment, the rotational motions kinetic profiles vary in accordance to a dosage changing activity of the injector such as dosage change, and/or an injection/dosage discharge. In some injectors, the rotational motions are about an axis, which is the axis of the rotation of the mechanical interface. The rotational axis of the mechanical interface can be parallel to the rotation of the dosage dial.

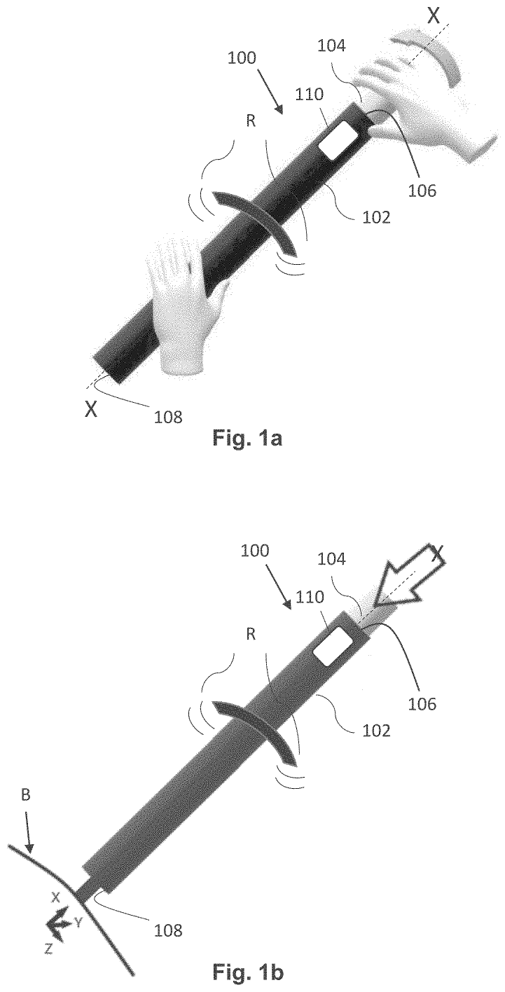

Referring now to FIGS. 1a and 1b, which are simplified illustrations (not proportional) of an injector 100, having rotational motions that can be detected according to some embodiments of the invention. Injector 100 has a mechanical interface producing rotational motions R in body 102 of injector 100 when changing the medicine dosage.

FIG. 1a shows an example of adjusting the dosage by using a rotatable dosage dial 104 located at a head injector end 106 of injector 100. In some embodiments, the rotation of dosage dial 104 is about an axis X and results in the rotational acceleration of injector body 102 about axis X. In some embodiments, the axis of rotation X of dosage dial 104 is longitudinal axis X of injector 100.

FIG. 1b shows an example of having rotational motions R during an injection operation into the patient body B. In some injectors, the dosage amount indicator reflects the dosage change during the injection operation.

### Detection System Components

According to some embodiments, the injector detection system is an external add-on sensing device connectable to the injector.

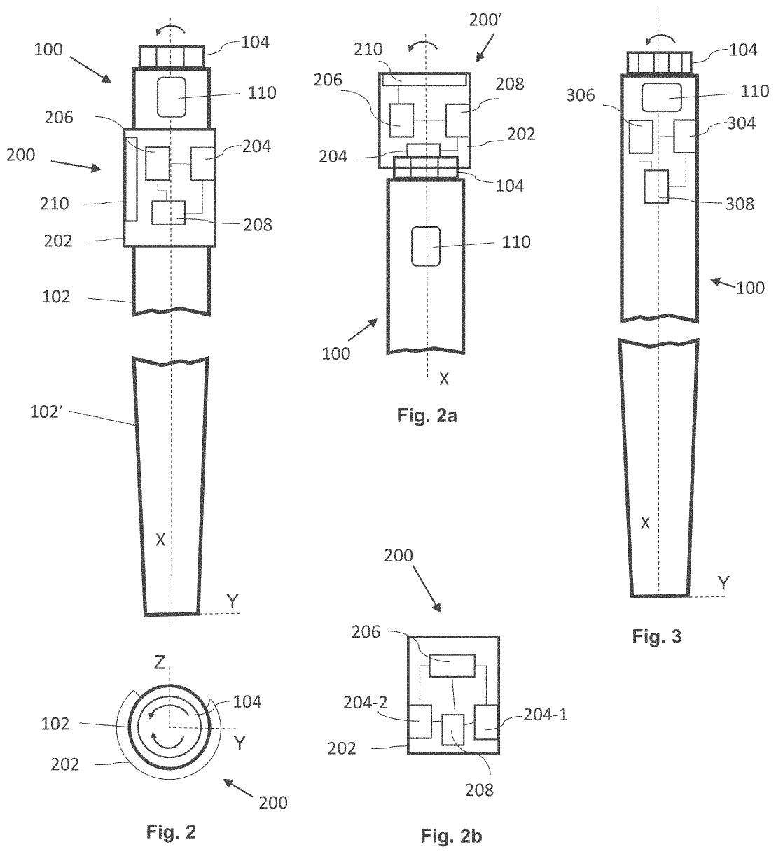

FIG. 2 shows an example of an add-on sensing device 200 that comprises a housing 202, connecting add-on sensing device 200 to injector 100.

Add-on sensing device 200 comprises one or more sensors 204 disposed within housing 202 to measure rotational motions of injector 100. One or more sensors 204 are structured and oriented for sensing the rotational motions R, when add-on sensing device 200 is connected to injector 100.

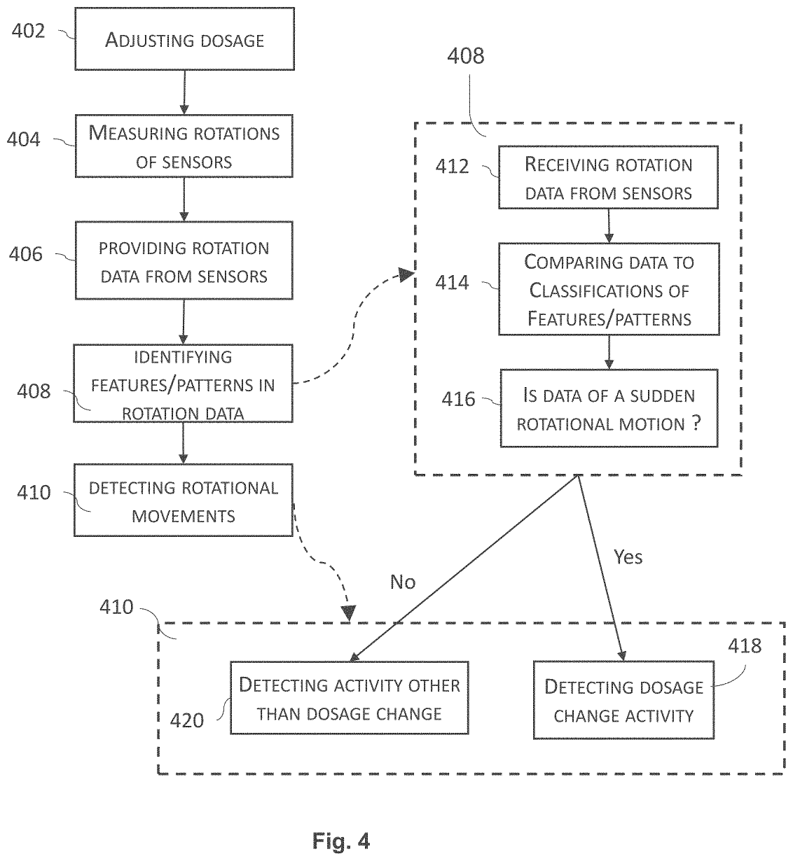

According to some embodiments, the system further comprises one or more processors 206. In some embodiments, processors 206 include a rotational data receiving functionality (e.g., element 414 in FIG. 4 described elsewhere herein), and a dosage change identification functionality (e.g., elements 416 and 418 in FIG. 4 described elsewhere herein) operatively connected thereto. The functionality (e.g. 414 to 418) of processor 206 can be implemented in various ways, such as software modules, functions, code sections, hardware, and combinations.

In operation, the rotational data receiving functionality receives rotational motions measurements data from sensors 204, and the dosage change identification functionality identifies in the rotational data, patterns corresponding to rotational motions produced by the mechanical coupling of the injector.

Turning to FIG. 2a that shows an example of an add-on sensing device 200′ that comprises a housing 202, configured to connect to dial 104 of injector 100 and one or more sensors 204 for measuring the rotational motions of the dial 104.

In some embodiment, during operation, sensing device 200′ is attached to dial 104, to have one or more sensors 204 engaging a face of dosage dial 104. In some embodiments, one or more sensors 204 are disposed in housing 202 and connecting sensing device 200′ to dial 104, does not engage sensors 204 with dial 104.

According to some embodiments, the injector accommodates at least one of the sensors, the processor, and/or other components of the detection system described elsewhere herein.

As shown in FIG. 3, in some embodiments, injector 100 accommodates one or more sensors 304 to measure rotational motions of injector 100. In some embodiments, sensors 304 are disposed on the mechanical coupling. In some embodiments, sensors 304 are connected to body 102.

As shown in FIG. 3, in some embodiments, injector 100 accommodates also processor 306, and a battery 308.

In some embodiments, an add-on device (e.g. 200/200′) comprises one or more of the detection system components, which are not included with injector 100.

### Identification and Detection Process

Referring to FIG. 4, which illustrates a diagram of a process for detecting activities of an injector, according to some embodiments of the invention. In the exemplified process shown in FIG. 4, the process comprises:

(402) adjusting dosage for example by increase dosage, decreasing dosage, or injecting a dosage.

(404) measuring rotations about axis X, using one or more sensors (e.g. 204/304);

(406) providing rotational data from sensors;

(408) identifying by processor (e.g. 206/306), one or more features and/or patterns in the rotational data; and

(410) detecting one or more activities of the injector according to the identified patterns.

The detection optionally depends on a classification of the patterns by dosage changing activities.

According to some embodiments, identifying 408 the processor is optionally:

(412) receiving (e.g. 206/306) rotations data from sensors (e.g. 204/306);

(414) comparing received data to classifications of features and/or patterns; and

(416) determining if data has the characteristic of a sudden rotational motion.

In some embodiments, detecting 410 is of activities, which are not dosage changing, such as tapping on the injector, and rolling of the injector. In some embodiments, these activities have patterns distinct of dosage changing. In some embodiments, these activities are detected using different sensors measurements.

In some embodiments, if determining 416 results of data that is of sudden rotational motion, detecting 418 is of a dosage change activity.

In some embodiments, if determining 416 results of data that is not of sudden rotational motion, detecting 420 is of activities, which are not dosage changing, such as tapping on the injector, and rolling of the injector. In some embodiments, these activities have patterns distinct of dosage changing. In some embodiments, these activities are detected using different sensors measurements.

According to some embodiments, measuring of rotation is about two or more axes. In some embodiments, the process includes defining noise in the rotational data by the measurements of rotations about axes other than X. In some embodiments, filter the rotational data is at least partially by the noise.

### Motion Features and Patterns

Features and patterns in the rotations recorded by the sensors can be associated with activities of the injector such as dosage change, dosage increase, dosage decrease, and injection.

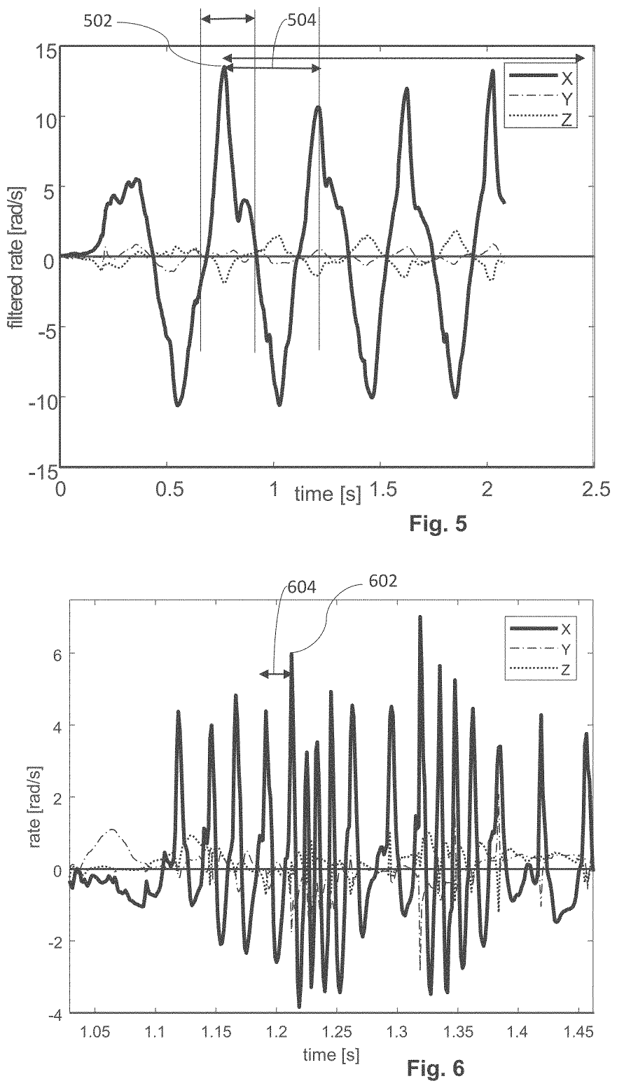

Turning to FIGS. 5 to 10, which are 2D graphs that illustrate example measurements of rotational movements of an injector about axes X, Y, and Z, according to some embodiments of the invention.

Rotational motions of the injector about axis X can be produced for example by rolling of the injector (e.g. for changing the orientation of the injector), or by the mechanical coupling between the dosage adjustment mechanism and the injector as described elsewhere herein.

The example measurements shown in FIGS. 5 and 6 demonstrate some of the differences between the profile of rotational motions of the injector due to manual rotation by human hand and rotational motions produced when increasing a dosage in a plurality of units.

FIG. 5 shows an example of measurements of rotations produced by manually rolling the injector about axis X in two directions.

Some of the features of the rotations in the example of FIG. 5 are rotational velocity peaks (e.g. 502) about the longitudinal axis X of injector 100 in the range of 5 to 15 rad/sec, and time between peaks (e.g. 504) in the range of 0.5 to 1 sec.

FIG. 6 shows an example of measurements of sudden rotations produced by the mechanical interface in response to dosage adjustment in a plurality of units of measure.

Some of the features of the rotations in the example of FIG. 6 are peaks of the rotational velocity (e.g. 602) about the longitudinal axis X of injector 100 in the range of 3 to 8 rad/sec, and time between peaks (e.g. 604) lower than 20 msec.

According to the example measurements shown in FIG. 5 and FIG. 6, the human hand does not roll the injector body in a rate as fast as rotational movements caused by the mechanical interface indicating a dosage change. For example, the rotational velocity of the mechanical interface can be at least 2 times lower that when manually rolling the injector. In some embodiments, the rotational velocity of the mechanical interface can be at least 5 times lower that when manually rolling the injector. In some embodiments, the rotational velocity of the mechanical interface can be at least 10 times lower that when manually rolling the injector.

Another difference is the time constants between rotational velocity peaks, which can be at least 5 times higher when manually rolling the injector compared than when produced by the mechanical interface.

According to some embodiments, a feature of motion as shown in FIG. 5, having a high time constant/low frequency indicates that a rotation of the injector body is a manual rolling of the body and is not a dosage change activity.

In some embodiments, a high time constant is higher than 0.2 sec. In some embodiments, a high time constant is higher than 0.5 sec. In some embodiments, a high time constant is higher than 1 sec.

According to some embodiments, a feature of motion having a low time constant/high frequency, as shown in FIG. 6, indicates that the rotation of the injector is a result of a plurality of dosage changes around axis X. In some embodiments, a low time constant is lower than 50 msec. In some embodiments, the low time constant is lower than 20 msec. In some embodiments, the low time constant is lower than 10 msec.

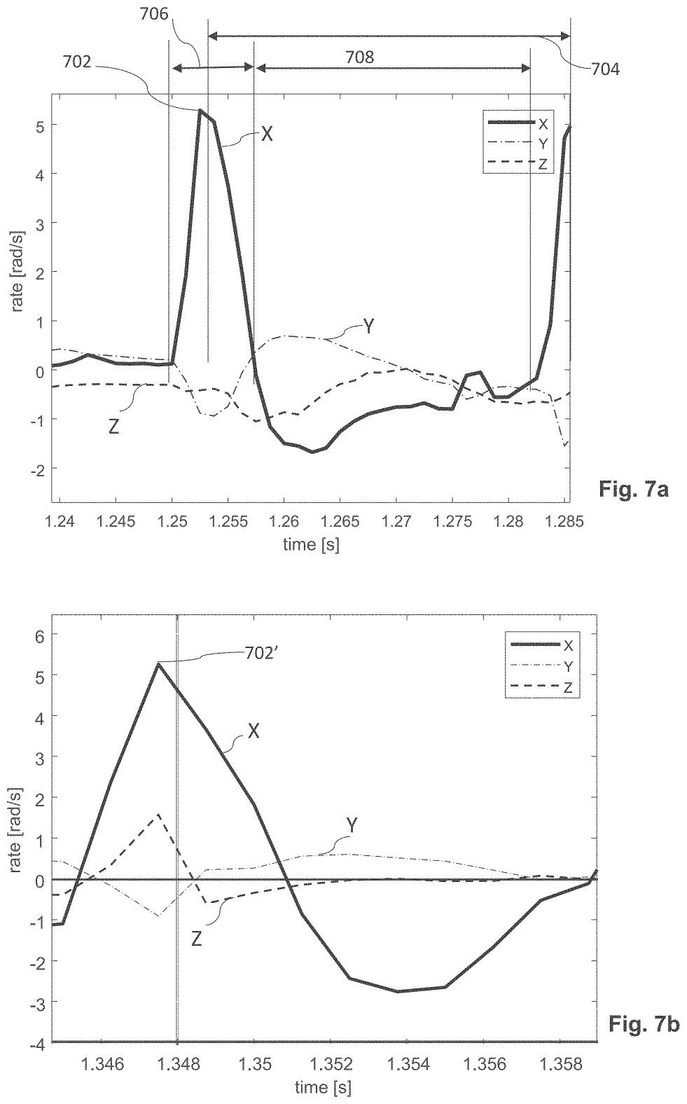

FIGS. 7a and 7b are examples of measurements of rotations, in two different injectors, produced by turning the dosage dial (around axis X) in a positive direction to increase the dosage, resulting in multiple positive rate peaks.

In the examples graphs 7a and 7b, the peak 702/702′ of the rotational motion rate measured about the longitudinal axis X of the injector is positive, followed by a negative rate.

According to embodiments, as in the examples of FIGS. 7a and 7b, a feature of the rotations is a positive rate peak value in the range of 5 to 6 rad/sec. This peak rate is higher than the peak rates of the rotational motions about the other axes Y and Z, which are lower than 2 rad/sec. In some embodiments, a positive peak rate is higher than 4 rad/sec. In some embodiments, a positive peak rate is higher than 6 rad/sec.

In some embodiments, a feature of the positive rotations is a time between the positive rate peaks 704 of about 10 to 80 msec. In some embodiments, a feature of the positive rotations is a time between the positive rate peaks 704 of about 20 to 60 msec. In some embodiments, a feature of the positive rotations is a time between the positive rate peaks 704 of about 30 to 50 msec.

In some embodiments, a feature of the positive rotations is a positive rate duration 706 of about 3 msec to 20 msec. In some embodiments, a feature of the positive rotations is a positive rate duration of about 5 msec to 10 msec. In some embodiments, a feature of the positive rotations is a positive rate duration of about 5 msec to 8 msec.

In some embodiments, a feature of the positive rotations is a negative rate duration 708 of about 5 msec to 20 msec. In some embodiments, a feature of the positive rotations is a negative rate duration of about 7 msec to 15 msec. In some embodiments, a feature of the positive rotations is a negative rate duration of about 8 msec to 12 msec.

In some embodiments, a feature of the positive rotations is a negative rate peak value of about 2 to 3 rad/sec. In some embodiments, a negative rate peak value is higher than 1 rad/sec in the negative direction. In some embodiments, a negative rate peak value is higher than 0.5 rad/sec in the negative direction.

In some embodiments, the ratio between a rate of a positive peak and to the rate of a negative peak during a positive dosage adjustment is higher than 1.3. In some embodiments, the ratio between a rate of a positive peak and to the rate of a negative peak during a positive dosage adjustment is higher than 2. In some embodiments, the ratio between a rate of a positive peak and to the rate of a negative peak during a positive dosage adjustment is higher than 5.

According to some embodiments of the invention, the positive rotation differs of the negative rotation by having a positive rate duration shorter than the duration of the negative rate 708. In some embodiments, the positive rotation differs of the negative rotation by having a negative rate peak value lower than the value of the positive peak.

In some embodiments, the integral of the change of the velocity is close to zero when there are rotational motions related to dosage change while there is no change in the orientation of the injector.

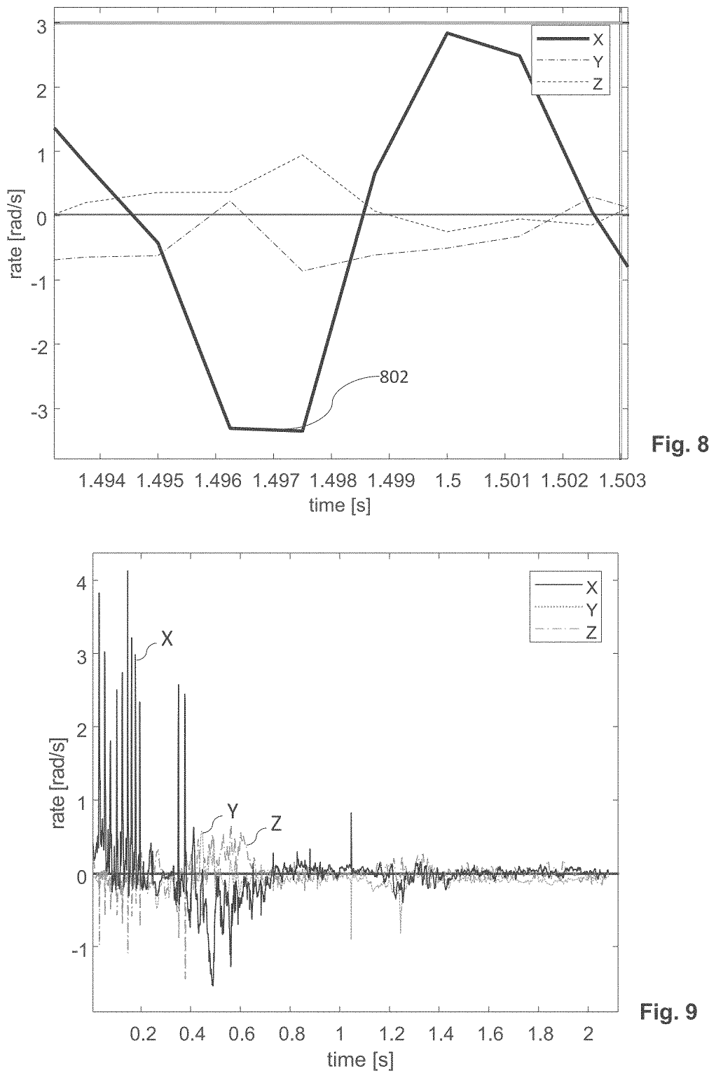

The example measurements shown in FIGS. 7a, 7b and FIG. 8 demonstrate that there is a different kinetic profile between rotational motions of the injector when increasing or decreasing a dosage.

FIG. 8 is an example of measurements of rotations produced by rotating the dosage dial (around axis X) in a negative direction to decrease the dosage in one unit of measures, resulting in a negative dosage change.

As shown in FIG. 8, the peak 802 of the rotational rate measured about the longitudinal axis X of the injector is in a negative rate followed by a positive rate, which is opposite to the rate directions of a positive dosage change as shown in FIGS. 7a to 7b. In some embodiments, the peak value of the negative rotational rate observed around the longitudinal axis X of the injector is about 3 rad/sec, which is higher than the peak value of the rotational motions rates about the other axes Y and Z (lower than 1 rad/sec).

According to some embodiments, during injection (or a discharge of dosage), the dosage decrease is faster than when manually decreasing the dosage by the dial. The mechanical interface mechanism produces rotational movements having an angular velocity profile that can be classified as having features and/or patterns of an injection activity.

FIG. 9 is an example of measurements of rotations produced by discharging a medicine dosage using an injection activity.

As shown in FIG. 9, in some embodiments, the peak value of the rotational rate observed around the longitudinal axis X of the injector is about 1-5 rad/sec, which is higher than the peak value of the rotational motions about the other axes Y and Z (lower than 1 rad/sec). In some embodiments, the time between peaks, when discharging a dose, is lower than the time between peaks observed when manually adjusting the dosage (e.g. FIG. 6). In some embodiments, the time between peaks the time between peaks, when discharging a dose, is lower than 0.05 sec. In some embodiments, the time between peaks, when discharging a dose, is lower than 0.03 sec. In some embodiments, the time between peaks, when discharging a dose, is lower than 0.02 sec.

According to some embodiments, a motion as shown in FIG. 9, having a feature of a low time constant/high frequency is an indication of an injection activity, and can be distinguished of a dosage adjustment. In some embodiments, a low time constant is defined as lower than 0.05 sec. In some embodiments, the low time constant is defined as lower than 0.03 sec. In some embodiments, the low time constant is defined as lower than 0.02 sec.

A potential advantage of having a detection system according to some embodiments of the present invention is that it can distinguish between a tapping on the injector, and activities related to dosage adjustment or injection.

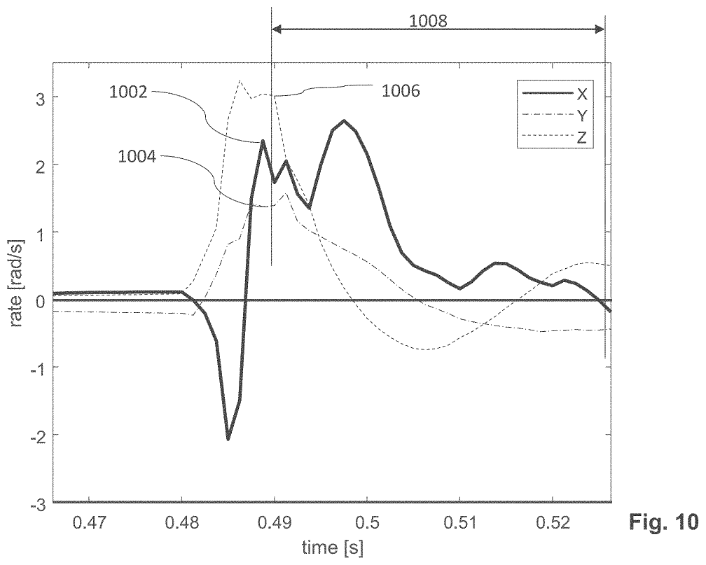

FIG. 10 is an example of measurements of rotations caused by tapping on the injector.

As shown in the example embodiment of FIG. 10, the sensor recorded tapping motions having rotational motions about the 3 axes X, Y, and Z with a rate in the same order of magnitude. In the example, the peak rate about axis X 1002 is about 2.5 rad/sec, peak rate about axis Y 1004 is about 1.5 rad/sec, and peak rate about axis Z 1006 is about 3 rad/sec. The peak rates about the 3 axes 1002, 1004, and 1006, occurred at about the same time, the tapping time.

In the example embodiment of FIG. 10, the signal decrease from the peak point 1002 is graduate, and the positive signal duration 1008 is shorter than when recording a dosage change (e.g. 704).

As shown in the examples of FIGS. 5 to 10, measuring rotations and identifying features and/or patterns in the rotations motions can be used to identify activities performed using the injector. In some embodiments, identifying is by comparing the value of the feature with a threshold. In some embodiments, identifying is by differentiating between feature and/or patterns of rotational movements and feature and/or patterns of linear movements. In some embodiments, identifying a motion as having a linear component is by having rotational movements about the first axis that do not have distinct features/patterns of rotational movements about the other one or two axes.

In some embodiments, identifying the direction of the rotations is by having an angular velocity higher than a threshold in one direction and an angular velocity lower than threshold in the opposite direction. In some embodiments, identifying an increasing the dosage is by having a pattern of peak angular velocity higher than a threshold, followed by a decrease in the angular velocity. In some embodiments, identifying a decreasing of the dosage by the rotation of the dosage dial is by having a pattern of peak angular velocity lower than a threshold, followed by an increase of the angular velocity.

According to some embodiments, defining a rotational pattern definition can include one or more of the features described elsewhere herein.

In some embodiments, a feature is having a maximal rotation rate (peak rate) about axis X higher than a threshold. Adding this feature can help identifying a positive dosage change (dosage increase). For example, maximal rotation rate of at least 2 rad/s about axis X.

In some embodiments, a feature is a duration of positive rotation rate. In some embodiments, a duration of positive rotation rate can be compared to a threshold for separating between dosage change and another activity having a high rotation rate, such as in rolling the injector. For example, having a maximal duration of 0.05 seconds, when the velocity about axis X is higher than 1 rad/s.

In some embodiments, a feature is a duration of return motion on the negative direction. Adding this feature can help separating between dosage change and tapping and/or rolling of the injector.

In some embodiments, a feature is a maximal rotation rate on the negative direction. In some embodiments, a maximal rotation rate on the negative direction is compared to a threshold. For example, a maximal rotation rate during the return (negative) motion is at least 1 rad/s. Adding this feature can help identifying a dosage change as a dosage decrease.

In some embodiments, a feature is a ratio between the maximal positive rotation rate and the return peak (maximal negative rotation rate). Adding this feature can help identifying a dosage change as a dosage increase. For example, a ratio between the maximal rotation rate and the return peak is at least 0.9.

In some embodiments, a feature is a return motion duration longer or shorter than the duration of the positive motion. A potential advantage of adding this feature is that it can help to identify a positive dosage adjustment and to distinguish between dosage increase and dosage decrease.

In some embodiments, a feature is a ratio between peak rate on X axis and peak rates on other axes. For example, a peak rotation rate on axes Y and/or Z is maximum 80% of the peak rate on X axis. A potential advantage of adding this feature is that it can help to distinguish between tapping on the injector, and activities related to dosage adjustment or injection. Another potential advantage of adding this feature is that it can help filtering noise according to the single strength of the rotations about axes Y and/or Z.

In some embodiments, a feature is time gaps between peaks in the rate of the rotational motions. For example, an absence of a dosage change in the last 5 milliseconds. A potential advantage of adding this feature is that it can help preventing recognizing one peak as two. Another potential advantage of adding this feature is that it can help identifying dosage discharge.

In some embodiments, a feature is time gaps between peaks in opposing directions. For example, an absence of a dosage change on the opposite direction of at least 50 milliseconds. A potential advantage of adding this feature is that it can improve identification of a dosage decrease, taking into account an assumption that a reaction of an operator is limited when changing rotation direction.

### Calibration of the Detection System

The kinetic profile of the sudden rotational movements produced by the mechanical interface vary by the structure of the injector, and by the operator of the device. Some of the varying factors of the injector can be materials, mechanical interface type, mechanical interface size, size of injector, etc. Some of the varying factors due to the operator can be speed of dial rotation, and pressure applied by the operator on the device.

In order to overcome the variations, calibration of the detection system might be required. Calibration might be required for example, when using different types of injectors, and/or when used by another operator. In some embodiments, calibration is in manufacturing. In some embodiments, calibration is by the user.

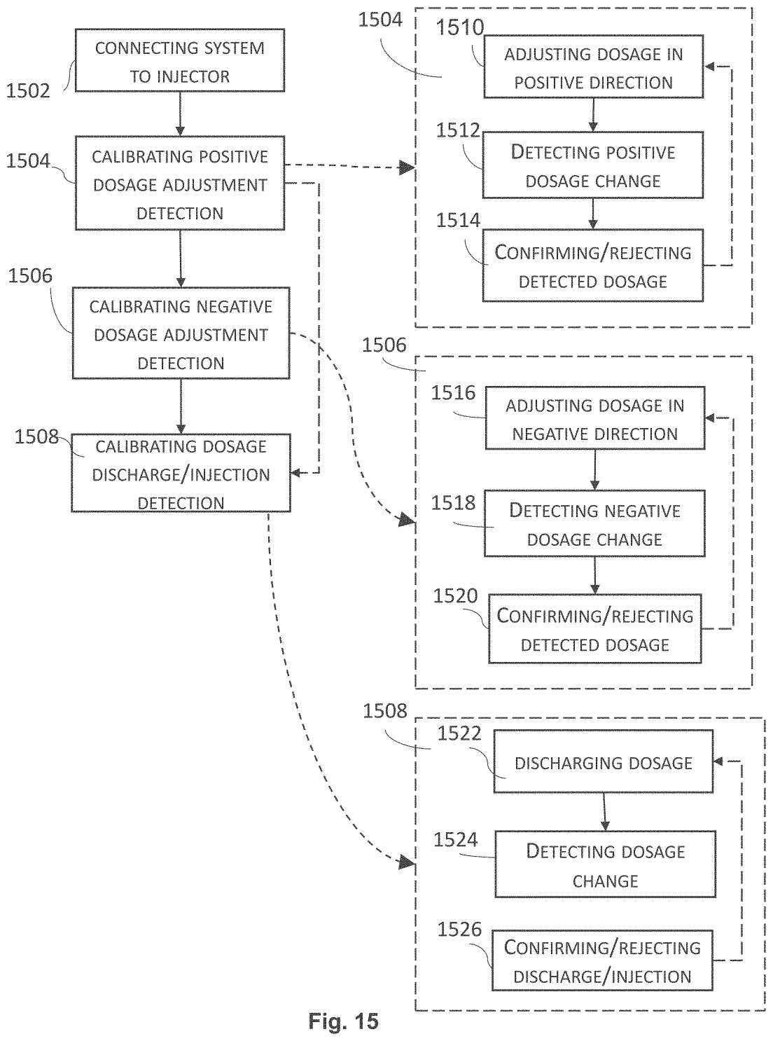

Referring to FIG. 15, which illustrates a diagram of a process for calibrating a detection system, according to some embodiments of the invention. In the exemplified process shown in FIG. 15, the calibration process comprises connecting detection system (e.g. 200) to the injector and activating the system 1502, and calibrating positive dosage adjustment detection 1504 by: adjusting dosage in a positive direction 1510, detecting positive dosage change by the detection system 1512, and confirming or rejecting dosage amount 1514. In some embodiments, calibrating positive dosage detection 1504 is repeated to receive positive confirmation of the detected dosage.

In some embodiments, calibration comprises calibrating negative dosage adjustment detection 1506 by: adjusting dosage in a positive direction 1516, detecting negative dosage change by the detection system 1518, and confirming or rejecting detected dosage amount 1520. In some embodiments, calibrating negative dosage detection 1506 is repeated to receive positive confirmation of the detected dosage. In some embodiments, calibrating negative dosage adjustment detection 1506, follows calibrating positive dosage adjustment detection 1504.

In some embodiments, calibration comprises calibrating dosage discharge detection 1508 by: discharging dosage 1522, detecting dosage discharge change by the detection system 1524, and confirming or rejecting a dosage discharge/injection 1526. In some embodiments, calibrating dosage discharge detection 1508 is repeated to receive positive confirmation of the detected discharge. In some embodiments, calibrating dosage discharge detection 1508 follows calibrating positive dosage adjustment detection 1504. In some embodiments, calibrating dosage discharge detection 1508 follows calibrating negative dosage adjustment detection 1506.

According to some embodiments, calibrating of the detection system comprises a machine-learning program. In some embodiments, the machine-learning program receive as an input kinetic features and/or patterns as described elsewhere herein, during calibration, and/or during routine usage of the injector. In some embodiments, adjusting of thresholds described elsewhere herein is by the machine-learning program.

In some embodiments, a detected amount is manually adjusted by the operator, by comparing dosage value detected by the detection device to a value displayed by the injector.

### Sensors Options

FIGS. 5 to 10 show examples of measuring rotations by a single gyro sensor. In some embodiments, at least one of sensors 204/304 is a gyro.

As shown in FIG. 2b, in some embodiments, measuring is by two or more sensors 204-1 and 204-2. In some embodiments, measuring the rotational motions is by two or more accelerometers 204-1 and 204-2 disposed at two side walls of the add-on sensing device 200.

The mechanical interface may produce “clicking” sounds that correspond to the sudden motion, by which the user receives a feedback about the increasing or the decreasing a medicine dose, and/or an injection. In some embodiments, the detection system comprises sound sensors (for example, one or more microphones) for detecting sound waves (clicks) generate by the mechanical interface discusses elsewhere herein.

In some embodiments, the positioning of sensor 204/304 on injector 100 affects the values of the motion signal received by sensor 204/304. In some embodiments, positioning the sensor closer to the mechanical coupling increases the signal strength. In some embodiments, the rotational motions signals are identifiable by sensor 204/304 at any location between injector head end 106 and injection needle end 108 of injector 100. In some embodiments, the rotational motions signals are identifiable at any location on the injector between injector head end 106 and the injection needle end 108 only when using a sensor for measuring angular rotation.

In some embodiments, a sensor is disposed at the mechanical coupling to measure the rotational movements of the mechanical coupling.

Damping of Rotational Motions Signals when Holding Injector

The quality of the detection of rotational movements may reduce when the rotation signal is damped. Some embodiments of the invention relate to preserving rotational motions than can be measured by the sensors, when applying pressure on its body, for example, when holding the injector.

In some embodiments, preserving the rotational motions is by controlling the pressure applied on the body of the injector, so the body can have rotational movements in response to motions of the mechanical coupling.

In some embodiments, preserving of the rotational motions is by maintaining the rotational movements of at least a portion of the body of the injector, while a pressure is applied on the injector.

In some embodiments, preserving of the rotational motions is by maintaining the rotational movements of at least a portion of the body of the injector, when applying a force higher than 0.5 on the body of the injector. In some embodiments, preserving of the rotational motions is by maintaining the rotational movements of at least a portion of the body of the injector, when applying a force higher than 1 Kg on the body of the injector.

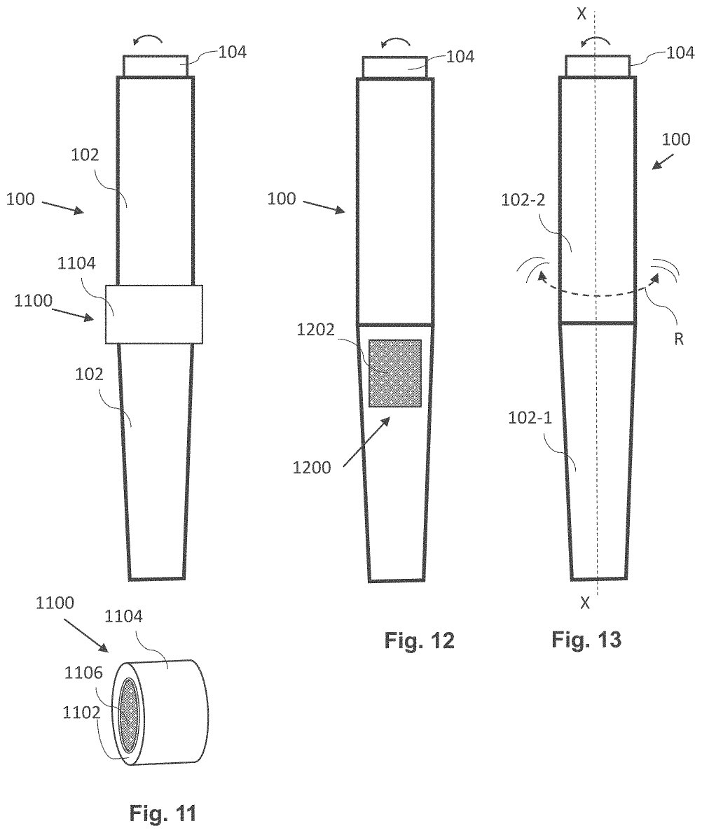

FIG. 11 is a simplified illustration of a detection system having a body holder for preserving the rotational motions of the injector when applying pressure on its body, according to an embodiment of the invention.

In the example embodiment of FIG. 11, the detection system (which can be any one of the add-on sensing devices described elsewhere herein) comprises a body holder 1100 having one or more cushion layers 1102 disposed at an internal surface of body holder 1100 and holding surface 1104 disposed at an external surface of body holder 1100. In operation, cushion layer 1102 engages, at least partially body 102, when connecting body holder 1100 to injector 100.

In operation, cushion layer 1102 reduces the damping of the rotational motions of body 102 in respect to holding surface 1104 when applying pressure by the operator on body holder 1100 to hold injector 100.

In some embodiments, the friction force between cushion layer 1102 and body 102 is high and the rotation between body 102 and holding surface 1104 produces shear rotational movements within cushion layer 1102.

In some embodiments, maintaining shear rotational movements within cushion layer 1102 is by having an elastic cushion layer 1102 under a pressure between holding surface 1104 and body 102.

In some embodiments, the ratio between the depth and the elastic coefficient of cushion layer 1102 is defined to preserve rotational shear movements of the elastic cushion layer 1102 in a maximal pressure applied on holding surface 1104.

In some embodiments, the friction force between cushion layer 1102 and body 102 is small and the rotation between body 102 and holding surface 1104 is by a rotational sliding of body 102 in respect to holding surface 1104.

As shown in FIG. 11, body holder 1100 can be cylindrical. In some embodiments, the body holder 1100 is not cylindrical. In some embodiments, the body holder 1100 is resilient. In some embodiments, cushion layer 1102 is composed of to a plurality of cushion layers 1102 disposed at an internal surface of body holder 1100.

Some embodiments related to having a body holder connected to add-on sensing device 200. In some embodiments, a body holder is connected to housing 202 of add-on 200.

FIG. 12 is a simplified illustration of an injector having a body holder on the body for preserving the rotational motions of the injector, according to an embodiment of the invention.

Body 102 of injector 100 comprises one or more external cushion layers 1200 embedded within body 102 of injector 100. In some embodiments, one or more external cushion layers 1200 are disposed around the circular surface of body 102.

Cushion layers 1200 optionally functions similarly to cushion layer 1102, as described above. An internal surface of the cushion layers 1200 is connected to body 102, and an external surface is a holding surface, which can be used to hold the injector 100.

In some embodiments, cushion layer 1102/1200 is made of an elastic material. In some embodiments, cushion layer 1102/1200 is made of a sponge like material.

FIG. 13 is a simplified illustration of a detection system having an injector built of one rotatable body and a second holding body, according to an embodiment of the invention.

According to some embodiments, injector 100 comprises a holding body 102-1 and a rotating body 102-2 so that rotating body 102-2 is rotatable when holding the holding body 102-1. In some embodiments, one or more sensors 204/304 measure the rotational motions of the rotating body 102-2. As shown in FIG. 13, rotating body 102-2 and holding body 102-1 can have a common longitudinal axis X, and the rotating body 102-2 is rotatable about axis X when holding body 102-1 is not rotating about axis X.

Some embodiments of the invention relate to indicating about a pressure applied on the body of the injector during its operation, which can result in the absorption of body motions and reduction in measuring rotational motions.

According to an embodiment of the invention, the system comprises a pressure sensor, which can optionally generate a signal indicating a pressure applied on the body of the injector during its operation. This signal may be identified as an excessive pressure, for example, by comparison to a threshold. In some embodiments, the pressure sensor is disposed at body 102 of injector 100. In some embodiments, the pressure sensor alerts about excessive pressure when identifying an unrecognized pattern of rotational motions. In some embodiments, the pressure sensor alerts about excessive pressure when identifying conflicting detections by two or more sensors.

### Housing Options

According to some embodiments, housing 202 is configured to be connected to body 102 of injector 100 for transmitting rotational motions of injector 100 to sensors within housing 202.

According to some embodiments, housing 202 is shaped to fit a plurality of pen-type injector types, having different shapes and/or sizes at the attachment area, and maintaining a transmission of rotational movements between body 102 and sensors 204. In some embodiments, housing 202 is shaped to fit pen-type injectors having a diameter at the attachment area in the range of 10 to 25 mm. In some embodiments, housing 202 fits pen-type injectors having a diameter at the attachment area in the range of 15 to 19 mm.

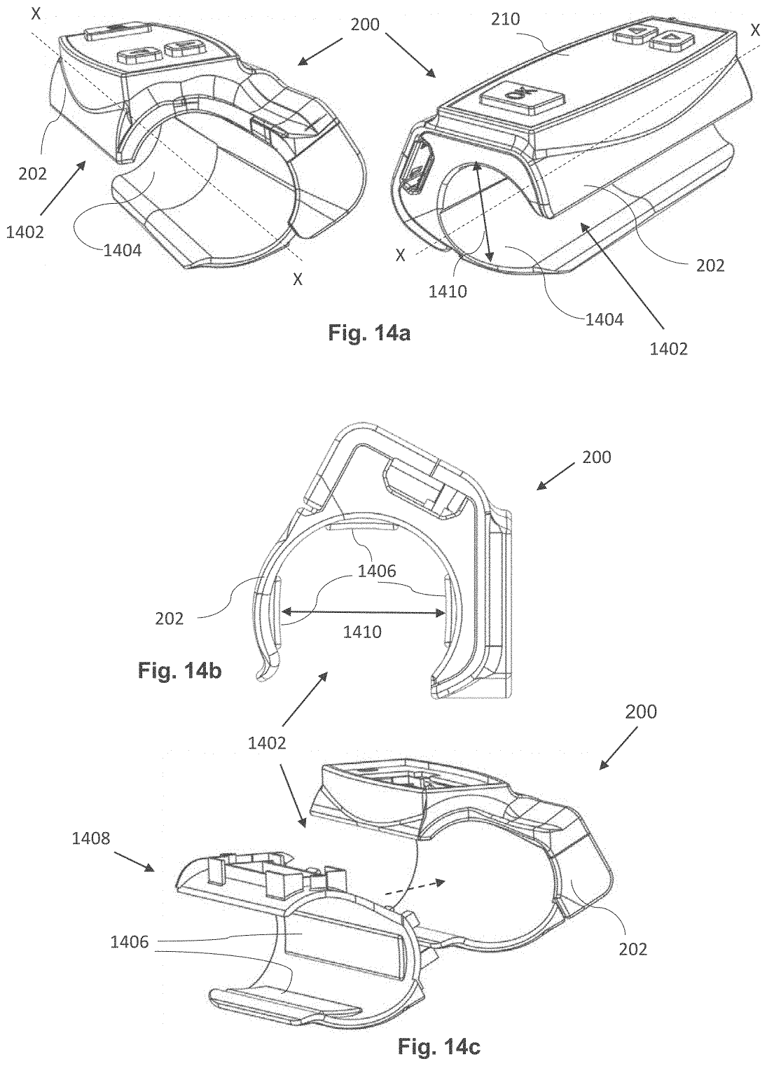

Turning to FIGS. 14a-14c, which are simplified illustrations of an add-on sensing device, according to some embodiments of the invention.

As shown in in FIG. 14a, add-on sensing device 200 comprises a housing 202 having an attachment opening 1402, and an inside contact surface 1404 for engaging the outside surface of body 102 of injector 100 (shown in FIG. 2).

In some embodiments, housing 202 is resilient and connecting housing 202 to injector 100 is by clamping housing 202 over injector body 102 through opening 1402. In some embodiments, housing 202 is sled over injector 100. In some embodiments, add-on sensing device 200 is detachable of body 102.

In some embodiments, the size and shape of contact surface 1404 is defined to urge inside contact surface 1404 on body 102 when add-on sensing device 200 is connected to injector 100.

In some embodiments, inside contact surface 1404 comprises an inside surface diameter 1410, and a relaxed state, in which housing 202 is not deployed on an injector and is free of external pressure. In some embodiments, when in relaxed state, diameter 1410 is equal or smaller than a diameter of an outside surface of body 102, on which housing 202 is configured to be attached. In some embodiments, diameter 1410 increases when attaching housing to body 102, and surface 1404 is urged against an outside attachment surface of body 102. In some embodiments, diameter 1410, in relaxed state, is smaller than outside attachment surface of body 102 in at least 0.01 mm. In some embodiments, diameter 1410, in relaxed state, is smaller than outside attachment surface of body 102 in at least 0.1 mm. In some embodiments, diameter 1410, in relaxed state, is smaller than outside attachment surface of body 102 in at least 1 mm. In some embodiments, diameter 1410, in relaxed state, is smaller than outside attachment surface of body 102 in at least 0.1%. In some embodiments, diameter 1410, in relaxed state, is smaller than outside attachment surface of body 102 in at least 1%. In some embodiments, diameter 1410, in relaxed state, is smaller than outside attachment surface of body 102 in at least 5%.

In some embodiment, a friction between inside contact surface 1404 and the outside surface of body 102 is used to maintain housing 202 in a fixed position in respect to body 102. In some embodiments, the friction coefficient between contact surface 1404 and the outside surface of body 102 is higher than 0.65. In some embodiment, the friction coefficient between contact surface 1404 and the outside surface of body 102 is higher than 0.80.

In some embodiments, fixing the position of housing 202 in respect to body 102 and maintaining a transmission of rotational movements between body 102 and sensors 204 is by defining the geometry of contact surface 1404 in accordance to the size and the geometry of one or more faces of the outside surface of body 102. In some embodiments, contact surface 1404 is cylindrical. In some embodiments, contact surface 1404 is conical. In some embodiments, contact surface 1404 has a varying cross-section along the length of housing 202.

In some embodiments, housing 202 comprising one or more protruding surfaces to match a corresponding protruding surfaces or slots at body 102 of injector 100. In some embodiments, housing 202 comprising one or more slots to match corresponding protruding surfaces at body 102 of injector 100.

In the example shown in FIG. 14b, housing 202 comprises one or more contact surfaces 1406 having a geometry that fits the geometry of body 102, preventing movement of housing 202 in respect to injector 100 and for maintaining a transmission of rotational movements between body 102 and sensors 204.

In the examples shown in FIG. 14c, housing 202 comprises a fitting 1408. Fitting 1408 comprises one or more protruding contact surfaces 1406 having a geometry that fits the geometry of body 102 to prevent movement of add-on 200 in respect to injector 100 and to maintain a transmission of rotational movements between body 102 and sensors 204.

In some embodiments, fitting 1408 comprising one or more slots to match a corresponding protruding surfaces at body 102 of injector 100.

One advantage of using a fitting 1408 is that housing 202 can be produced as a generic housing, while fitting 1408 is added to the generic housing to ensure a tight contact between one or more surfaces 1406 of add-on 200 and body 102.

In some embodiments, add-on sensing device 200 is reusable. In some embodiments, add-on sensing device 200 is attachable and detachable at least 50 times. In some embodiments, add-on sensing device 200 is disposable after being attached to an injector 100.

### Optional Configurations of the Add-on Sensing Device

As shown in FIGS. 2 and 14a, add-on sensing device 200 can comprise a display 210. Display 210 provides input to the operator about parameters measured by add-on device 200. For example, dosage units, time, alerts, etc.

In some embodiments, add-on sensing device 200 comprises control panel for controlling some activities of add-on device 200. In some embodiments, the panel comprises one or more buttons. For example, activating the device, adjusting the displayed dosage units, setting/turning off alerts, and setting time.

In some embodiments, the add-on device 200 comprises a panel for correcting dosage values detected by the device.

In some embodiments, add-on sensing device 200 comprises a communication circuit having an antenna for wireless transmission (e.g. Bluetooth, Wi-Fi).

In some embodiments, add-on sensing device 200 comprises a power source, such as a battery. In some embodiments, add-on sensing device 200 comprises a charging connector for charging the battery.

According to some embodiments, the detection system described herein is for insulin injection pens. In some embodiments, the system is for injectors of medicine other than insulin.

### Remote Components

In some embodiments, the add-on sensing device accommodates the processor. In some embodiments, a device other than the add-on sensing device accommodates the processor. In some embodiments, the processor receives sensor measurements through a wireless communication. In some embodiments, the processor is a handheld device. In some embodiments, detecting of the patterns as activities of the injector is using an application of the handheld device.

According to some embodiments, at least one of the detection system components is disposed remotely of injector 100 and add-on sensing device 200/200′.

In some embodiments (not shown), processor 206/306 is detached of add-on sensing device 200 and injector 100. In some embodiments, processor 206/306 receives measurements from sensor 204/306 through a wireless communication. In some embodiments, processor 206 is included within a handheld device.

In some embodiments, identifying patterns in rotational data is by an application of the handheld device. In some embodiments, detecting the rotational movements as dosage changing activity is by an application of the handheld device.

### General

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A method for detecting rotational movements due to dosage changing activity in a pen-type injector having a body and a dosage adjustment mechanism, comprising:
measuring, using a sensor, rotation about one or more axes of said injector to provide rotational data;
identifying, in said rotational data, one or more features and/or patterns corresponding to a change in the angular velocity of said sensor due to mechanical coupling between said body and said dosage adjustment mechanism; and
detecting said rotational movements based on said identifying.

2. A method according to claim 1, wherein said change in the angular velocity is a sudden change, having a sudden angular acceleration.

3. A method according to claim 1, wherein said one or more features comprise rotational data about a first axis having an angular velocity profile that is distinct from angular velocity profile about the other axes.

4. A method according to claim 1, comprising classifying said one or more features as dosage change by having an angular velocity higher than a threshold during an increase of the velocity or at the peak velocity.

5. A method according to claim 1, comprising classifying said one or more features as dosage change by having a positive angular velocity duration lower than a threshold.

6. A method according to claim 1, comprising classifying said one or more patterns as dosage increase by having a positive peak angular velocity higher than a threshold followed by a decreasing of the angular velocity to a negative rate.

7. A method according to claim 1, comprising classifying said one or more patterns as dosage decrease by having a peak negative angular velocity lower than a threshold followed by an increase in the angular velocity to a positive rate.

8. A method according to claim 1, comprising classifying said one or more features an injection operation by having a positive angular velocity duration lower than a threshold.

9. A method according to claim 1, comprising differentiating between patterns of rotational movements and patterns of linear movements by classifying a pattern of linear movements as absent of distinct rotational movements about one axis.

10. A method according to claim 1, comprising classifying said one or more patterns as a dosage adjustment by a ratio between a peak positive angular velocity to a peak negative angular velocity.

11. A method according to claim 1, wherein said measuring is about one axis.

12. A method according to claim 1, wherein said measuring is by one or more sensors disposed at an add-on sensing device connected to said injector.

13. A method according to claim 1, wherein said one or more sensors comprise a gyro sensor.

14. (canceled)

15. A dosage change indication system for an adjustable pen-type injector having a mechanical coupling between a dosage adjustment mechanism and the injector, comprising:
one or more sensors orientated to measure rotations about one or more axes of said injector, and to provide rotational data; and
one or more processors having a rotational data receiving functionality, and a dosage adjustment identification functionality operatively connected thereto to identify in said rotational data, one or more features and/or patterns, corresponding to a change in the angular velocity of said sensor due to said mechanical coupling.

16. A system according to claim 15, wherein said one or more sensors are configured to measure rotational movements about an axis, which is the axis of said rotational movements produced by said mechanical coupling.

17. A system according to claim 15, comprising an add-on sensing device having one or more connection surfaces, and said one or more sensors are disposed within said sensing device,
wherein the one or more connection surfaces are urged against said injector, when said add-on sensing device is connected to said injector.

18. A system according to claim 15, wherein said injector comprises a body and a dosage dial, and said add-on sensing device comprises a housing connected to said body, when said sensing device is connected to said injector.

19. (canceled)

20. A system according to claim 17, wherein said add-on sensing device comprises said processor.

21. A system according to claim 15, wherein said system comprises a wireless communication circuit and said processor receives sensor measurements through wireless communication.

22. A system according to claim 15, wherein said one or more sensors comprise a gyro sensor.

23-25. (canceled)
